(19) United States
(12) Patent Application Publication
(10) Pub. No.: US 2019/0388970 A1
(43) Pub. Date: Dec. 26, 2019

(54) ONE-STEP MANUFACTURING METHOD OF LAMINATED MOLDING POROUS COMPONENT WHICH HAS CURVED SURFACE

(71) Applicant: KOREA INSTITUTE OF INDUSTRIAL TECHNOLOGY, Cheonan-si (KR)

(72) Inventors: Kang Min Kim, Seoul (KR); Gun Hee Kim, Incheon (KR); Byoung Soo Lee, Gangneung-si (KR); Hyung Giun Kim, Gangneung-si (KR); O Hyung Kwon, Gangneung-si (KR); Kyung Hwan Jung, Daejeon (KR); Won Rae Kim, Gangneung-si (KR); Min Ji Ham, Gangwon-do (KR); Kyung Hoon Kim, Daejeon (KR); Chang Woo Lee, Seoul (KR)

(73) Assignee: KOREA INSTITUTE OF INDUSTRIAL TECHNOLOGY, Cheonan-si (KR)

(21) Appl. No.: 16/158,492

(22) Filed: Oct. 12, 2018

(30) Foreign Application Priority Data
Jun. 20, 2018 (KR) 10-2018-0070825

(51) Int. Cl. B22F 3/11; B22F 3/105; A61L 27/56; A61F 2/30; A61L 27/06

(52) U.S. Cl. CPC B22F 3/11; B22F 3/105; A61L 27/56; B33Y 10/00; A61L 27/06; A61F 2002/30985; A61F 2310/00023; A61F 2/30771

(57) ABSTRACT

An exemplary embodiment provides a method of manufacturing a curved porous component having a base material layer and a porous region through one-step laminated-molding, whereby it is possible to reduce a manufacturing time when manufacturing a product and to provide a porous component in which the shape and size of a porous region can be controlled. An implant including the porous component has an increased bone contact ratio, so bone growth between bones can be improved and products fitting to the frames of patients can be easily designed.

### BACKGROUND OF THE INVENTION

### Field of the Invention

The present invention relates to a one-step manufacturing method of laminated molding porous component which has a curved surface and, more particularly, to a method of manufacturing a curved porous component having a base material layer and a porous region through one step using a laminated molding technology to a process of manufacturing a porous component for increasing a bone contact ratio of an implant.

### Description of the Related Art

An implant means a material that is used when reconstructing a shape or substituting for a function by implanting an artificial material or a natural material in a lost portion to compensate for a loss of a biological tissue. In general, an implant means a biological material for substituting for hard tissues of a human body in dentistry or orthopedics, and studies related to dental implants have been actively conducted since the mid-1960s.

Metallic materials having high strength and hardness and low biological toxicity are selected as the materials of implants. In particular, titanium and titanium alloys, which are materials having excellent biocompatibility, have been known as having not only good biocompatibility for surrounding tissues, but large resistance against corrosion and little biological toxicity. For this reason, in the early stage of the study related to implants, titanium or titanium alloys were used as implants through simple machining.

An implant can be implanted to a lost portion only when it has compatibility to an existing biological tissue, so most implants are coated with a biological tissue adhesive on the surfaces. In particular, bone cement that is an adhesive inducing quick regeneration of a bone tissue has been used for complex fracture restoration and artificial joint operations that frequently occur due to traffic accidents etc. in the field of orthopedics and for dentin restoration of non-regenerative teeth in dentistry.

However, bioactive substances coated on the surfaces are dissolved too fast, and high temperature is generated in the coating process which makes it difficult to expect the effect of coated materials. Further, it has been reported that substances coming off coating layers may interfere with bonding of bones or may cause side effects such as inflammation.

In order to solve this problem, there has been proposed a method of coating an implant with a porous structure on the surface to improve growth of bones even without cement, and products using this method have been released.

However, this method also have a problem with bonding between an implant and a porous structure, and it is required to add a process of manufacturing a separate porous structure and then attaching it to an implant, which reduces productivity and increases the manufacturing costs of implants.

3D printing that has been recently actively studied may be an alternative measure that can solve the problem. It is possible to laminated-mold metallic materials such as titanium that is generally used as the material of implants, using 3D printing, so it may be possible to develop a new implant using this method.

### SUMMARY OF THE INVENTION

In order to solve the problems, an object of the present invention is to provide a method of manufacturing a curved porous component having a base material layer and a porous region through one step laminated molding.

Another object of the present invention is to provide a method of reducing a process time and controlling the shape and size of a porous region when manufacturing a product including a curved porous component.

The technical object to implement in the present invention are not limited to the technical problems described above, and other technical objects that are not stated herein will be clearly understood by those skilled in the art from the following specifications.

In order to achieve the objects, an embodiment of the present invention provides a one-step manufacturing method of laminated molding porous component which has a curved surface, the method including the steps of: layering metallic particles; forming a first base material layer having a curved edge by repeatedly melting and cooling the metallic particles by radiating a laser to the layered metallic particles; forming a first porous region by radiating a laser while adjusting a point distance to form laser radiation points having a predetermined diameter D on the metallic particles layered on the outer side of the curved edge of the first base material layer; layering metallic particles, which are the same as the metallic particles, on the first base material layer and the first porous region; forming a second base material layer having a curved edge by repeatedly melting and cooling the metallic particles layered on the first base material layer by radiating a laser to the metallic particles; and forming a second porous region by radiating a laser and adjusting point distances to form laser radiation points having a predetermined diameter D on the metallic particles layered on the outer side of the curved edge of the second base material layer.

In an embodiment of the present invention, the length of the curved edge of the second base material layer may be smaller than or same as the length of the curved edge of the first base material layer.

In an embodiment of the present invention, the laser radiation points in the step of forming the second porous region may be arranged not to overlap the laser radiation points on the first porous region.

In an embodiment of the present invention, the metallic particles may be one or more selected from a group of titanium (Ti), a titanium (Ti)-based alloy, cobalt (Co), a cobalt (Co)-based alloy, nickel (Ni), a nickel (Ni)-based alloy, zirconium (Zr), a zirconium (Zr)-based alloy, barium (Ba), a barium (Ba)-based alloy, magnesium (Mg), a magnesium (Mg)-based alloy, vanadium (V), a vanadium (V)-based alloy, iron (Fe), an iron (Fe)-based alloy, and mixture of them.

In an embodiment of the present invention, the laser may have energy equal to or greater than complete melting energy of the metallic particles in the step of forming a first base material layer and in the step of forming a second base material layer.

In an embodiment of the present invention, in the step of forming a first porous region and in the step of forming a second porous region, the laser has energy equal to or greater than 0.2 times the complete melting energy within a range equal to or less than the complete melting energy of the metallic particles.

In an embodiment of the present invention, the point distance may be greater than the diameter D of the laser radiation points in the step of forming a first porous region and in the step of forming a second porous region.

In an embodiment of the present invention, the diameter D of the laser radiation points may be in proportion to source power and exposure time of the laser and the exposure time may be in inverse proportion to the scan speed of the laser.

In an embodiment of the present invention, the source power of the laser may be 50 W to 1 KW, and the scan speed may be 0.1 m/s to 8 m/s.

In an embodiment of the present invention, the point distance may be 100 to 1000 μm.

In order to achieve the objects, another embodiment of the present invention provides a laminated molding porous component which has a curved surface and formed by the method.

In order to achieve the objects, another embodiment of the present invention provides an implant having an increased bone contact ratio and including the porous component.

### BRIEF DESCRIPTION OF THE DRAWINGS

FIG. 1 is a flowchart showing a one-step manufacturing method of laminated molding porous component which has a curved surface;

FIG. 2 is a vertical cross-sectional view of a porous component which has a curved surface according to the present invention;

FIG. 3 is a horizontal cross-sectional view of a porous component which has a curved surface according to the present invention;

FIG. 4 is a picture showing a laser radiation method when forming a base material layer according to the present invention; and

FIG. 5 is a picture showing a laser radiation method when forming a porous region according to the present invention.

### DETAILED DESCRIPTION OF THE PREFERRED EMBODIMENTS

Hereinafter, the present invention is described with reference to the accompanying drawings. However, the present invention may be modified in various different ways and is not limited to the embodiments described herein. Further, in the accompanying drawings, components irrelevant to the description will be omitted in order to obviously describe the present invention, and similar reference numerals will be used to describe similar components throughout the specification.

Throughout the specification, when an element is referred to as being “connected with (coupled to, combined with, in contact with)” another element, it may be “directly connected” to the other element and may also be “indirectly connected” to the other element with another element intervening therebetween. Further, unless explicitly described otherwise, “comprising” any components will be understood to imply the inclusion of other components rather than the exclusion of any other components.

Terms used in this specification are used only in order to describe specific exemplary embodiments rather than limiting the present invention. Singular forms are intended to include plural forms unless the context clearly indicates otherwise. It will be further understood that the terms “comprise” or “have” used in this specification, specify the presence of stated features, numerals, steps, operations, components, parts, or a combination thereof, but do not preclude the presence or addition of one or more other features, numerals, steps, operations, components, parts, or a combination thereof.

Hereinafter, embodiments of the present invention are described in detail with reference to the accompanying drawings.

A one-step manufacturing method of laminated molding porous component which has a curved surface is described hereafter.

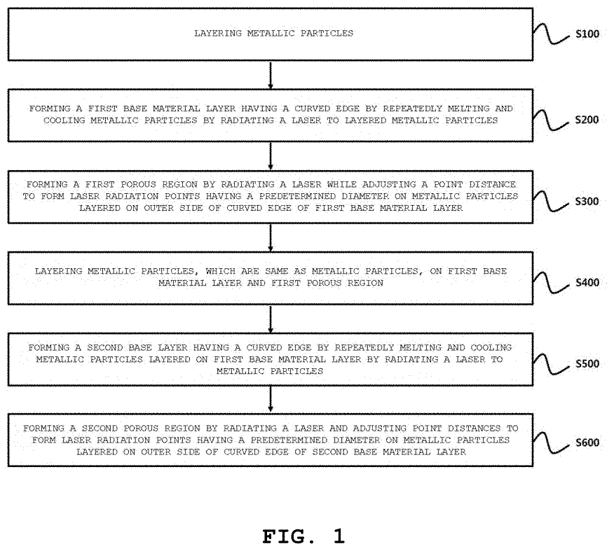

Referring to FIG. 1, an embodiment of the present invention provides a one-step manufacturing method of laminated molding porous component which has a curved surface, the method including the steps of: layering metallic particles (S100); forming a first base material layer having a curved edge by repeatedly melting and cooling the metallic particles by radiating a laser to the layered metallic particles (S200); forming a first porous region by radiating a laser while adjusting a point distance to form laser radiation points having a predetermined diameter D on the metallic particles layered on the outer side of the curved edge of the first base material layer (S300); layering metallic particles, which are the same as the metallic particles, on the first base material layer and the first porous region (S400); forming a second base material layer having a curved edge by repeatedly melting and cooling the metallic particles layered on the first base material layer by radiating a laser to the metallic particles (S500); and forming a second porous region by radiating a laser and adjusting point distances to form laser radiation points having a predetermined diameter D on the metallic particles layered on the outer side of the curved edge of the second base material layer (S600).

The porous component which has a curved surface of the present invention may have a shape of which the cross-sectional area is gradually decreased upward from the bottom like a hemisphere or a shape of which the cross-sectional area is uniform from the bottom to the top like a cylinder. The porous component which has a curved surface is not limited to the shapes and has only to be decreased or uniform in cross-sectional area from the bottom to the top, and the shape of the edge is not limited. The edge may be a curved surface, and molding is possible even if the edge is formed in a polygonal shape or a star shape composed of several straight lines. However, in the case of the shape of which the cross-sectional area increases upward, machinability is good when it is machined in a shape of which the cross-sectional area decreases upward. Complicated shapes that repeatedly increase and decrease in cross-sectional area make machinability poor.

The length of the curved edge of the second base material layer may be smaller than or the same as the length of the curved edge of the first base material layer.

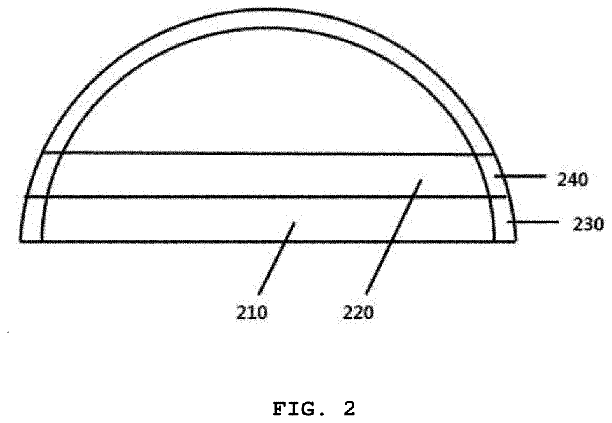

FIG. 2 is a vertical cross-sectional view of a porous component which has a curved surface according to the present invention. FIG. 2 shows an exemplary vertical cross-section of a semispherical porous component, in which a second base material layer 220 is formed on a first base material layer 210. A first porous region 230 is on the outer side of the edge of the first base material layer 210, and a second porous region 240 is on the outer side of the edge of the second base material layer 220. To help understanding, the first base material layer 210 and the second base material layer 220 are shown thicker than real. The first porous region 230 and the second porous region 240 are also shown thicker than real.

The first base material layer 210 is formed first by layering metallic particles and then radiating a laser, the first porous region 230 is then formed on the outer side of the edge, the second base material layer 220 is formed by layering metallic particles again on the first base material layer and the first porous region and then by radiating a laser, and then the second porous region 240 is formed on the outer side of the edge.

The laser radiation points in the step of forming the second porous region may be arranged not to overlap the laser radiation points on the first porous region.

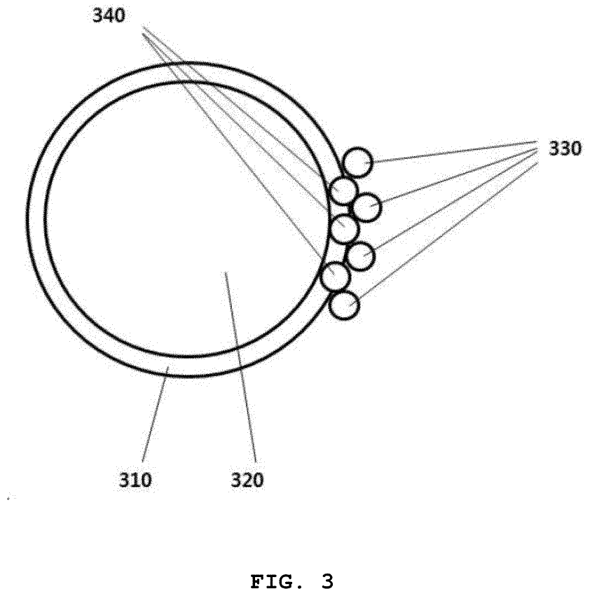

FIG. 3 is a horizontal cross-sectional view of a porous component which has a curved surface according to the present invention. FIG. 3 shows an exemplary horizontal cross-section of a semispherical porous component, in which a second base material layer 320 is formed on a first base material layer 310. A first porous region 330 is on the outer side of the edge of the first base material layer 310, and a second porous region 340 is on the outer side of the edge of the second base material layer 320. To help understanding the thickness difference between the first base material layer 310 and the second base material layer 320 and the sizes of the first porous region 330 and the second porous region 340 are shown larger than real.

The first porous region 330 is formed by radiating a laser while adjusting a point distance to form a laser radiation point having a predetermined diameter D on the metallic particles layered on the outer side of the curved edge of the first base material layer 310. The second porous region 340 is formed by radiating a laser while adjusting a point distance to form a laser radiation point having a predetermined diameter D on the metallic particles layered on the outer side of the curved edge of the second base material layer 320. As shown in FIG. 3, laser radiation points in the second porous region are arranged not to overlap the laser radiation points in the first porous region 330. A porous structure can be formed by the non-overlapping arrangement, and the first porous region 330 and the second porous region 340 may be adjacent to each other even though the laser radiation points do not overlap one another. The adjacent structure is advantages in terms of securing strength because it forms continuous porous regions.

The metallic particles may be one or more selected from a group of titanium (Ti), a titanium (Ti)-based alloy, cobalt (Co), a cobalt (Co)-based alloy, nickel (Ni), a nickel (Ni)-based alloy, zirconium (Zr), a zirconium (Zr)-based alloy, barium (Ba), a barium (Ba)-based alloy, magnesium (Mg), a magnesium (Mg)-based alloy, vanadium (V), a vanadium (V)-based alloy, iron (Fe), an iron (Fe)-based alloy, and mixture of them.

In particular, titanium and titanium-based alloys, which are materials having excellent biocompatibility, have been known as having not only good biocompatibility for surrounding tissues, but large resistance against corrosion and little biological toxicity, so they are preferable. However, the present invention is not limited thereto and the metallic particles described above can be selectively used.

The laser may have energy equal to or greater than complete melting energy of the metallic particles in the step of forming the first base material layer and the step of forming the second base material layer.

In the steps of forming the first porous region and forming the second porous region, the laser may have energy equal to or greater than 0.2 times the complete melting energy within a range equal to or less than the complete melting energy of the metallic particles.

When energy greater than the complete melting energy is applied to the metallic particles, the metallic particles may be completely melted and densified. When smaller energy is applied to the metallic particles, the metallic particles may be formed in a porous type without being densified.

That is, when forming base material layers and porous regions in the present invention, the base material layers can be densified by inputting energy equal to or greater than the complete melting energy and the porous regions can be formed in porous type by inputting energy equal to or greater than 0.2 times the complete melting energy within a range equal to or less than the complete melting energy. The porosity is another factor that forms a porous structure separate from radiating a laser while adjusting a point distance when forming laser radiation points. When the laser has energy less than 0.2 times the complete melting energy of the metallic particles, the metallic particles are never melted, so it is not preferable.

The point distance may be greater than the diameter D of the laser radiation points in the step of forming the first porous region and the step of forming the second porous region.

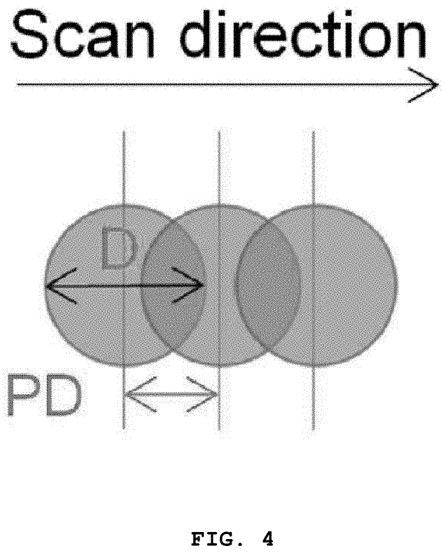

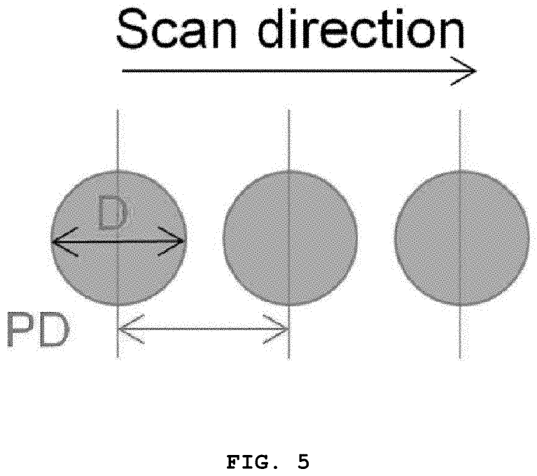

Referring to FIGS. 4 and 5, a manner of radiating a laser in the present invention can be seen. FIG. 4 shows a laser radiation manner in common laminated-molding. A laser is radiated to a base material layer in the manner shown in FIG. 4 in the present invention. The point distance PD becomes smaller than the diameter D of the laser radiation points, so the laser radiation points partially overlap one another. FIG. 5 shows a laser radiation manner when forming a porous region in the present invention, in which the point distance PD becomes larger than the diameter D, so the laser radiation point does not overlap each other. Accordingly, metallic particles are melted only at the laser radiation points and a porous structure is formed.

The diameter D of the laser radiation points is in proportion to the source power and exposure time of the laser and the exposure time may be in inverse proportion to the scan speed of the laser.

The source power of the laser may be 50 W to 1 KW, and the scan speed may be 0.1 m/s to 8 m/s.

The conditions of the source power and the scan speed may depend on the kind of metallic particles and the structure of a porous region to be formed. For example, when a base material layer that requires high-density molding is formed using pure titanium, energy of 5.5 to 6.5 J or more per cubic millimeters should be provided, and this can be achieved in conditions of the source power of 100 W or more at a scan speed of 0.25 m/s.

Energy equal to or less than the complete melting energy can be radiated when a porous region is formed, so the source power can be reduced at the same scan speed. Further, it is also possible to increase the scan speed with the source power maintained in order to increase the laser radiation point distance. However, when the scan speed is increased too much, the exposure time of a laser may be decreased and the diameter of the laser radiation points may become too small, so it is preferable to adjust the scan speed within the range described above.

The point distance may be 100 to 1000 μm. When the point distance is less than 100 μm, the diameter D of laser radiation points that should be smaller than the point distance is too small, so machinability is deteriorated. When the point distance exceeds 1000 μm, the diameter D of laser radiation points should be correspondingly increased to be able to form a porous region, and for this purpose, the laser source power should also be increased, so it is not preferable. Further, when the point distance exceeds 1000 μm, there is another problem that the specific surface area of the porous region is small.

The present invention further provides a laminated-molding porous component which has a curved surface that is manufactured by the method. The laminated-molding porous component which has a curved surface according to the present invention has an integrated base material layer-porous region, so the manufacturing time is reduced and the manufacturing process is simple in comparison to existing products formed using porous coating.

The present invention further provides an implant having an increased bone contact ratio and including the porous component. The porous component according to the present invention has many pores having a diameter of 100 to 1000 μm, so it has improved bone contact ratio and bone growth in comparison to implants using a biological tissue adhesive such as bone cement. Further, since the porous region is integrally formed, an implant that is more excellent in strength and durability can be provided.

The present invention is described in more detail hereafter with reference to a preferred embodiment. However, it should be noted that the present invention is not limited thereto and the embodiment is just an example.

### EMBODIMENT

Pure titanium particles were layered and a circular first base material layer was formed by radiating a laser at a scan speed of 0.5 m/s and source power of 200 W. A first porous region was formed by radiating a laser to the pure titanium particles layered around the first base material layer, with point distances of 350 μm to form laser radiation points having a diameter of 70 μm. A circular second base material layer was formed by layering pure titanium particles again on the first base material layer and the first porous region and then radiating a laser under the same condition as that for the first base material layer. The diameter of the second base material layer was smaller by 50 μm than that of the first base material layer. A second porous region was formed by radiating a laser to the pure titanium particles layered around the second base material layer, with point distances of 350 μm to form laser radiation pints having a diameter of 70 μm.

The following Table 1 shows laser radiation conditions when forming the first porous region and the second porous region in the embodiment.

TABLE 1

| | Scan | Source | Exposure |
|---|---|---|---|
| | speed | power | time |
| Items | (m/s) | (W) | (μs) |

| | | | | |
|---|---|---|---|---|
| Embodiment | First porous | 0.875 | 200 | 400 |
| | region | | | |
| | Second porous | 0.875 | 200 | 400 |
| | region | | | |

When a porous region is formed in accordance with the method of manufacturing a porous component which has a curved surface of the present invention, laser radiation conditions such as a scan speed, source power, and exposure time are set in accordance with the kind of metallic particles and the structure of a porous region which has a curved surface to be formed, whereby it is possible to easily design implants fitting to the frames of patients.

According to an embodiment of the present invention, it is possible to reduce a manufacturing time when manufacturing a product using one-step laminated-molding, and it is also possible to provide a porous component which has a curved surface in which the shape and size of a porous region can be controlled.

Further, an implant including the porous component which has a curved surface has an increased bone contact ratio, so bone growth between bones can be improved and products fitting to the frames of individual patients can be easily designed.

The effects of the present invention are not limited thereto and it should be understood that the effects include all effects that can be inferred from the configuration of the present invention described in the following specification or claims.

The above description is provided as an exemplary embodiment of the present invention and it should be understood that the present invention may be easily modified in other various ways without changing the spirit or the necessary features of the present invention by those skilled in the art. Therefore, the embodiments described above are only examples and should not be construed as being limitative in all respects. For example, single components may be divided and separate components may be integrated.

The scope of the present invention is defined by the following claims, and all of changes and modifications obtained from the meaning and range of claims and equivalent concepts should be construed as being included in the scope of the present invention.

### REFERENCE SIGNS LIST

210, 310: first base material layer
220, 320: second base material layer
230, 330: first porous region
240, 340: second porous region

What is claimed is:

1. A one-step manufacturing method of laminated molding porous component which has a curved surface, the method including the steps of:
layering metallic particles;
forming a first base material layer having a curved edge by repeatedly melting and cooling the metallic particles by radiating a laser to the layered metallic particles;
forming a first porous region by radiating a laser while adjusting a point distance to form laser radiation points having a predetermined diameter D on the metallic particles layered on the outer side of the curved edge of the first base material layer;
layering metallic particles, which are the same as the metallic particles, on the first base material layer and the first porous region;
forming a second base material layer having a curved edge by repeatedly melting and cooling the metallic particles layered on the first base material layer by radiating a laser to the metallic particles; and
forming a second porous region by radiating a laser and adjusting point distances to form laser radiation points having a predetermined diameter D on the metallic particles layered on the outer side of the curved edge of the second base material layer.

2. The method of claim 1, wherein the length of the curved edge of the second base material layer is smaller than or the same as the length of the curved edge of the first base material layer.

3. The method of claim 1, wherein the laser radiation points in the step of forming a second porous region are arranged not to overlap the laser radiation points on the first porous region.

4. The method of claim 1, wherein the metallic particles are one or more selected from a group of titanium (Ti), a titanium (Ti)-based alloy, cobalt (Co), a cobalt (Co)-based alloy, nickel (Ni), a nickel (Ni)-based alloy, zirconium (Zr), a zirconium (Zr)-based alloy, barium (Ba), a barium (Ba)-based alloy, magnesium (Mg), a magnesium (Mg)-based alloy, vanadium (V), a vanadium (V)-based alloy, iron (Fe), an iron (Fe)-based alloy, and mixture of them.

5. The method of claim 1, wherein the laser has energy equal to or greater than complete melting energy of the metallic particles in the step of forming a first base material layer and in the step of forming a second base material layer.

6. The method of claim 1, wherein in the step of forming a first porous region and in the step of forming a second porous region, the laser has energy equal to or greater than 0.2 times the complete melting energy within a range equal to or less than the complete melting energy of the metallic particles.

7. The method of claim 1, wherein the point distance is greater than the diameter D of the laser radiation points in the step of forming a first porous region and in the step of forming a second porous region.

8. The method of claim 7, wherein the diameter D of the laser radiation points is in proportion to source power and exposure time of the laser and the exposure time is in inverse proportion to the scan speed of the laser.

9. The method of claim 8, wherein the source power of the laser is 50 W to 1 KW and the scan speed is 0.1 m/s to 8 m/s.

10. The method of claim 7, wherein the point distance is 100 to 1000 μm.

11. A laminated-molding porous component which has a curved surface and formed by the method of claim 1.

12. An implant having an increased bone contact ratio and including the porous component of claim 11.
